(19) United States
(12) Patent Application Publication
(10) Pub. No.: US 2022/0223287 A1
(43) Pub. Date: Jul. 14, 2022

(54) AI BASED SYSTEM AND METHOD FOR PREDICITING CONTINUOUS CARDIAC OUTPUT (CCO) OF PATIENTS

(71) Applicant: Praveen Koduru, The Woodlands, TX (US)

(72) Inventor: Praveen Koduru, The Woodlands, TX (US)

(21) Appl. No.: 17/700,558

(22) Filed: Mar. 22, 2022

Related U.S. Application Data
(63) Continuation-in-part of application No. 14/877,756, filed on Oct. 7, 2015, now abandoned.

(51) Int. Cl. G16H 50/20; G16H 50/70; G16H 10/60

(52) U.S. Cl. CPC G16H 50/20; G16H 10/60; G16H 50/70

(57) ABSTRACT

A system and method for predicting the Continuous Cardiac Output (CCO) of patients is disclosed. The method includes receiving a request from one or more patients and one or medical professionals and determining a set of recovery patterns of the one or more patients. The method further classifying the one or more patients into one or more predefined profiles and predicting the CCO of the one or more patients based on the request, the set of recovery patterns and the result of classification by using health management based AI model. Further, the method includes generating one or more medical recommendations corresponding to the predicted CCO and outputting the predicted CCO of the one or more patients and the generated one or more medical recommendations on user interface screen of one or more electronic devices associated with the one or more patients and the one or more medical professionals.

### CROSS-REFERENCE TO RELATED APPLICATIONS

This application is a continuation in part of a non-provisional patent application filed in the US having patent application Ser. No. 14/877,756 filed on Oct. 7, 2015 and titled “METHOD AND SYSTEM FOR PREDICTING CONTINUOUS CARDIAC OUTPUT (CCO) OF A PATIENT BASED ON PHYSIOLOGICAL DATA”.

### FIELD OF INVENTION

Embodiments of the present disclosure relate to patient monitoring systems and more particularly relates to a system and a method for predicting Continuous Cardiac Output (CCO) of patients.

### BACKGROUND

Generally, prognosis of patients during recovery relies on monitoring and analyzing various physiological data, such as heart rate, central venous pressure and the like, that is collected over time to analyze and identify potential problems ahead of time. Especially in Intensive Care Unit (ICU), the physiological data becomes invaluable and hence the patients are continuously monitored on various vital signs for providing proactive care.

Further, the patients are continuously monitored on various physiological data and vital signs during their post-surgery recovery in the ICU. Cardiac output i.e., the volumetric rate at which blood is pumped through the heart, is one of the most important cardiovascular parameters. The cardiac output reflects supply of oxygen and nutrients to tissues of the patient. Measurements of the cardiac output provides invaluable clinical information for quantifying extent of cardiac dysfunction, indicating optimal course of therapy, managing patient progress, and establishing check points for rehabilitation in the patient with a damaged or diseased heart, or one in whom fluid status control is essential. Exercise, as well as pathological conditions of the heart and circulatory system may alter cardiac output; therefore, measurement of the cardiac output is useful both in rehabilitation and critically ill patients.

Conventionally known continuous, non-invasive method for measuring the cardiac output is based on measurement of body impedance. In impedance-cardiographic measurement, electrodes are placed on upper part of the patient's body, and the impedance between the electrodes is measured. The electrical impedance thus measured shows cyclic changes due to cardiac activity, allowing cardiac output to be calculated on the basis of theoretic models and empiric formulas. Impedance measurement has the advantage of simplicity, and that it allows continuous, fast and non-invasive measurement of the cardiac output. However, a significant drawback with the conventional method is its inaccuracy and inability to forecast into the future because these models are simple empirical formulas based on correlation factors and assumptions that are not sufficient for accurate prediction.

Hence, there is a need for an improved AI based system and method for predicting Continuous Cardiac Output (CCO) of patients ahead of time, in order to address the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

In accordance with an embodiment of the present disclosure, an AI based computing system for predicting Continuous Cardiac Output (CCO) of patients is disclosed. The AI based computing system includes one or more hardware processors and a memory coupled to the one or more hardware processors. The memory includes a plurality of modules in the form of programmable instructions executable by the one or more hardware processors. The plurality of modules include a data receiver module configured to receive a request from at least one of: one or more patients and one or more medical professionals to predict CCO associated with the one or more patients. The received request includes physiological data of the one or more patients. The plurality of modules also include a pattern determination module configured to determine a set of recovery patterns of the one or more patients based on the received request and one or more responses of the one or more patients to a treatment regime by using a health management based Artificial Intelligence (AI) model. The plurality of modules includes a patient classification module configured to classify the one or more patients into one or more predefined profiles based on the received request and the determined set of recovery patterns by using the health management based AI model. Each of the one or more predefined profiles corresponds to a set of patients with similar recovery patterns. Further, the plurality of modules includes a data prediction module configured to predict the CCO of the one or more patients based on the received request, the determined set of recovery patterns and the result of classification by using the health management based AI model. The plurality of modules also include a recommendation generation module configured to generate one or more medical recommendations corresponding to the predicted CCO based on the received request, the determined set of recovery patterns, the result of classification and the predicted CCO by using the health management based AI model. The one or more medical recommendations correspond to supply of oxygen and nutrients to tissue of the one or more patients, extent of cardiac dysfunction, optimal course of therapy, patient progress management, check points for rehabilitation in patient with one of: damaged and diseased heart and fluid status control. Furthermore, the plurality of modules include a data output module configured to output the predicted CCO of the one or more patients and the generated one or more medical recommendations on user interface screen of one or more electronic devices associated with the one or more patients and the one or more medical professionals.

In accordance with another embodiment of the present disclosure, an AI based method for predicting Continuous Cardiac Output (CCO) of patients is disclosed. The AI based method includes receiving a request from at least one of: one or more patients and one or more medical professionals to predict CCO associated with the one or more patients. The received request includes physiological data of the one or more patients. The AI based method also includes determining a set of recovery patterns of the one or more patients based on the received request and one or more responses of the one or more patients to a treatment regime by using a health management based Artificial Intelligence (AI) model. The AI based method further includes classifying the one or more patients into one or more predefined profiles based on the received request and the determined set of recovery patterns by using the health management based AI model. Each of the one or more predefined profiles corresponds to a set of patients with similar recovery patterns. Further, the AI based method includes predicting the CCO of the one or more patients based on the received request, the determined set of recovery patterns and the result of classification by using the health management based AI model. Also, the AI based method includes generating one or more medical recommendations corresponding to the predicted CCO based on the received request, the determined set of recovery patterns, the result of classification and the predicted CCO by using the health management based AI model. The one or more medical correspond to supply of oxygen and nutrients to tissue of the one or more patients, extent of cardiac dysfunction, optimal course of therapy, patient progress management, check points for rehabilitation in patient with one of: damaged and diseased heart and fluid status control. Furthermore, the AI based method includes outputting the predicted CCO of the one or more patients and the generated one or more medical recommendations on user interface screen of one or more electronic devices associated with the one or more patients and the one or more medical professionals.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF DRAWINGS

The disclosure will be described and explained with additional specificity and detail with the accompanying figures in which:

FIG. 1 is a block diagram illustrating an exemplary computing environment for predicting Continuous Cardiac Output (CCO) of patients, in accordance with an embodiment of the present disclosure;

FIG. 2 is a block diagram illustrating an exemplary AI based computing system for predicting CCO of patients, in accordance with an embodiment of the present disclosure;

FIG. 3 is a process flow diagram illustrating an exemplary AI based method for predicting CCO of patients, in accordance with an embodiment of the present disclosure;

FIG. 4 is an exemplary graphical representation illustrating a sample time series for comparing nearest neighbour interpolation and linear interpolation to represent missing data replacement, in accordance with an embodiment of the present disclosure;

FIG. 5 is an exemplary plot diagram illustrating a health management based AI model prediction of CCO 10 minutes into future based on input training data, in accordance with an embodiment of the present disclosure;

FIG. 6 is an exemplary plot diagram illustrating a prediction of CCO 10 minutes into future with testing data to validate the health management based AI model, in accordance with an embodiment of the present disclosure;

FIG. 7 is an exemplary plot diagram illustrating the health management based AI model prediction of CCO 30 minutes into future based on the input training data, in accordance with another embodiment of the present disclosure;

FIG. 8 is an exemplary plot diagram illustrating prediction of CCO 30 minutes into future by the health management based AI model with the testing data to validate the health management based AI model, in accordance with another embodiment of the present disclosure;

FIG. 9 is an exemplary plot diagram illustrating the health management based AI model prediction of CCO 60 minutes into future based on the input training data, in accordance with an embodiment of the present disclosure; and

FIG. 10 is an exemplary plot diagram illustrating prediction of CCO 60 Minutes into future by the health management based AI model with the testing data to validate the health management based AI model, in accordance with an embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client or server computer system) configured by an application may constitute a “module” (or “subsystem”) that is configured and operated to perform certain operations. In one embodiment, the “module” or “subsystem” may be implemented mechanically or electronically, so a module include dedicated circuitry or logic that is permanently configured (within a special-purpose processor) to perform certain operations. In another embodiment, a “module” or “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired) or temporarily configured (programmed) to operate in a certain manner and/or to perform certain operations described herein.

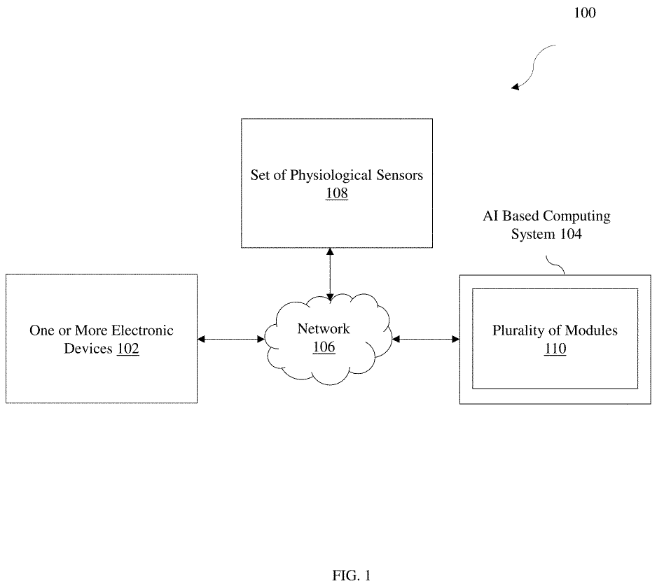

Referring now to the drawings, and more particularly to FIG. 1 through FIG. 10, where similar reference characters denote corresponding features consistently throughout the figures, there are shown preferred embodiments and these embodiments are described in the context of the following exemplary system and/or method.

FIG. 1 is a block diagram illustrating an exemplary computing environment 100 for predicting Continuous Cardiac Output (CCO) of patients, in accordance with an embodiment of the present disclosure. According to FIG. 1, the computing environment 100 includes one or more electronic devices 102 associated with one or more patients and one or more medical professionals communicatively coupled to an AI based computing system 104 via a network 106. The one or more electronic devices 102 are used by one or more patients and one or more medical professionals to request the AI based computing system 104 to predict Continuous Cardiac Output (CCO) of the one or more patients and generate one or more medical recommendations corresponding to the predicted CCO. In an embodiment of the present disclosure, the one or more medical recommendations correspond to supply of oxygen and nutrients to tissue of the one or more patients, extent of cardiac dysfunction, optimal course of therapy, patient progress management, check points for rehabilitation in patient with damaged or diseased heart, fluid status control and the like. The one or more electronic devices 102 are also used by the one or more patients and the one or more medical professionals to receive the predicted CCO of the one or more patients and the generated one or more medical recommendations. In an exemplary embodiment of the present disclosure, the one or more electronic devices 102 may include a laptop computer, desktop computer, tablet computer, smartphone, wearable device, smart watch and the like. Further, the network 106 may be internet or any other wireless network. The AI based computing system 104 may be hosted on a central server, such as cloud server or a remote server.

Further, the computing environment 100 includes a set of physiological sensors 108 communicatively coupled to an AI based computing system 104 via the network 106. The set of physiological sensors 108 are configured to capture physiological data of the one or more patients. The set of physiological sensors 108 include Electrocardiogram (ECG) sensor, blood pressure sensor, temperature sensor, heart rate sensor, blood glucose sensor and the like. In an exemplary embodiment of the present disclosure, the physiological data of the one or more patients includes Arterial Pressures (AR), Heart Rate (HR), Central Venous Pressure (CVP), Pulmonary Artery Pressure (PAP), Peripheral capillary oxygen saturation (SpO2), Mixed venous oxygen saturation (SvO2), Core Body Temperature (CBT), Continuous Systemic Vascular Resistance (CSVR) and the like.

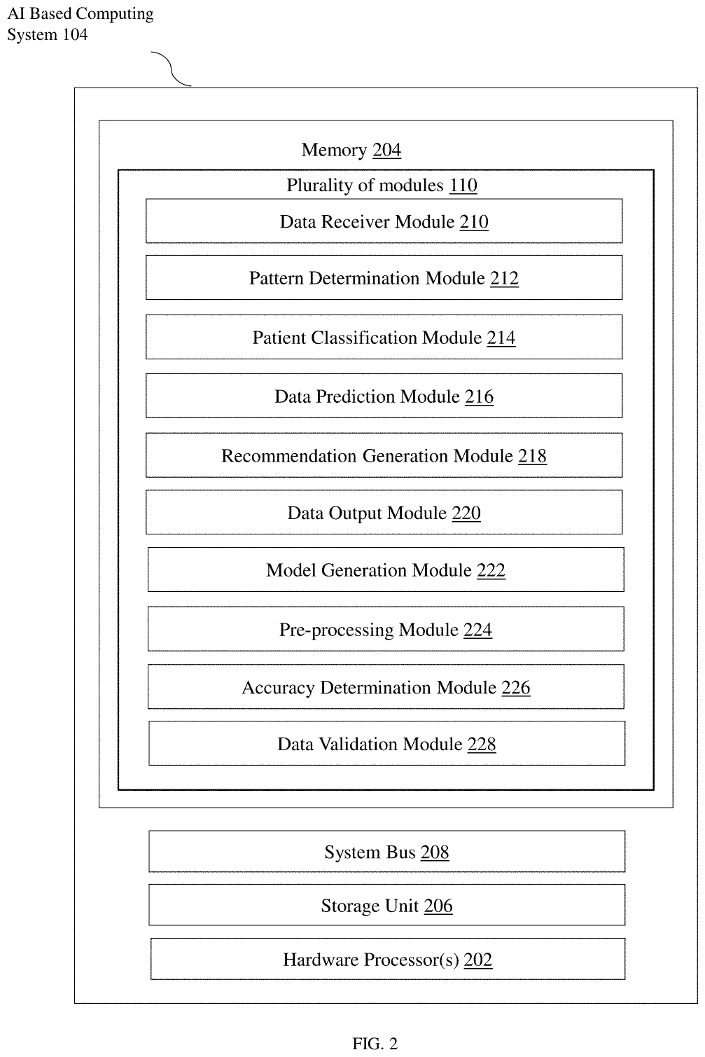

Furthermore, the one or more electronic devices 102 include a local browser, a mobile application or a combination thereof. Furthermore, the one or more patients and the one or more medical professionals may use a web application via the local browser, the mobile application or a combination thereof to communicate with the AI based computing system 104. In an embodiment of the present disclosure, the AI based computing system 104 includes a plurality of modules 110. Details on the plurality of modules 110 have been elaborated in subsequent paragraphs of the present description with reference to FIG. 2.

In an embodiment of the present disclosure, the AI based computing system 104 is configured to receive a request from the one or more patients, the one or medical professionals or a combination thereof to predict the CCO associated with the one or more patients. The received request includes the physiological data of the one or more patients. Further, the AI based computing system 104 determines a set of recovery patterns of the one or more patients based on the received request and one or more responses of the one or more patients to a treatment regime by using a health management based Artificial Intelligence (AI) model. The AI based computing system 104 classifies the one or more patients into one or more predefined profiles based on the received request and the determined set of recovery patterns by using the health management based AI model. The AI based computing system 104 predicts the CCO of the one or more patients based on the received request, the determined set of recovery patterns and the result of classification by using the health management based AI model. The AI based computing system 104 generates one or more medical recommendations corresponding to the predicted CCO based on the received request, the determined set of recovery patterns, the result of classification and the predicted CCO by using the health management based AI model. Further, the AI based computing system 104 outputs the predicted CCO of the one or more patients and the generated one or more medical recommendations on user interface screen of one or more electronic devices 102 associated with the one or more patients and the one or more medical professionals.

FIG. 2 is a block diagram illustrating an exemplary AI based computing system 104 for predicting Continuous Cardiac Output (CCO) of patients, in accordance with an embodiment of the present disclosure. Further, the AI based computing system 104 includes one or more hardware processors 202, a memory 204 and a storage unit 206. The one or more hardware processors 202, the memory 204 and the storage unit 206 are communicatively coupled through a system bus 208 or any similar mechanism. The memory 204 comprises the plurality of modules 110 in the form of programmable instructions executable by the one or more hardware processors 202. Further, the plurality of modules 110 includes a data receiver module 210, a pattern determination module 212, a patient classification module 214, a data prediction module 216, a recommendation generation module 218, a data output module 220, a model generation module 222, a pre-processing module 224, an accuracy determination module 226 and a data validation module 228.

The one or more hardware processors 202, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 202 may also include embedded controllers, such as generic or programmable logic devices or arrays, application specific integrated circuits, single-chip computers, and the like.

The memory 204 may be non-transitory volatile memory and non-volatile memory. The memory 204 may be coupled for communication with the one or more hardware processors 202, such as being a computer-readable storage medium. The one or more hardware processors 202 may execute machine-readable instructions and/or source code stored in the memory 204. A variety of machine-readable instructions may be stored in and accessed from the memory 204. The memory 204 may include any suitable elements for storing data and machine-readable instructions, such as read only memory, random access memory, erasable programmable read only memory, electrically erasable programmable read only memory, a hard drive, a removable media drive for handling compact disks, digital video disks, diskettes, magnetic tape cartridges, memory cards, and the like. In the present embodiment, the memory 204 includes the plurality of modules 110 stored in the form of machine-readable instructions on any of the above-mentioned storage media and may be in communication with and executed by the one or more hardware processors 202.

The storage unit 206 may be a cloud storage. The storage unit may store the received request, the set of recovery patterns and the one or more responses of the one or more patients to the treatment regime. The storage unit 206 may also store the Continuous Cardiac Output (CCO) of the one or more patients and the one or more medical recommendations.

The data receiver module 210 is configured to receive the request from the one or more patients, the one or medical professionals or a combination thereof to predict the CCO associated with the one or more patients. For example, the one or more medical professionals may be physician, nurse and the like. In an embodiment of the present disclosure, the received request includes the physiological data of the one or more patients. The physiological data of the one or more patients include Arterial Pressures (AR), Heart Rate (HR), Central Venous Pressure (CVP), Pulmonary Artery Pressure (PAP), Peripheral capillary oxygen saturation (SpO2), Mixed venous oxygen saturation (SvO2), Core Body Temperature (CBT), Continuous Systemic Vascular Resistance (CSVR) and the like. In an embodiment of the present disclosure, the one or more patients are ICU patients who have undergone cardiac surgery. The one or more patients are monitored continuously, and the physiological data is collected on a minute-by-minute basis during their recovery to normality under medical supervision in the ICU. Further, the physiological data is captured by the set of physiological sensors 108, such as Electrocardiogram (ECG) sensor, blood pressure sensor, temperature sensor, heart rate sensor, blood glucose sensor and the like. The AR may correspond to Systolic, Diastolic and Mean. In an embodiment of the present disclosure, the request may be received from the one or more electronic devices 102 associated with the one or more patients and the one or more medical professionals. In an exemplary embodiment of the present disclosure, the one or more electronic devices 102 may include a laptop computer, desktop computer, tablet computer, smartphone, wearable device, smart watch, and the like.

The pattern determination module 212 is configured to determine the set of recovery patterns of the one or more patients based on the received request and the one or more responses of the one or more patients to a treatment regime by using the health management based Artificial Intelligence (AI) model.

The patient classification module 214 is configured to classify the one or more patients into the one or more predefined profiles based on the received request and the determined set of recovery patterns by using the health management based AI model. In an embodiment of the present disclosure, each of the one or more predefined profiles corresponds to a set of patients with similar recovery patterns.

The data prediction module 216 is configured to predict the CCO of the one or more patients based on the received request, the determined set of recovery patterns and the result of classification by using the health management based AI model. In an embodiment of the present disclosure, the CCO of the one or more patients is physiological parameter of the one or more patients. The data prediction module 216 predicts the CCO of the one or more patients ahead of time. In an embodiment of the present disclosure, the CCO of the one or more patients corresponds to CCO level of the one or more patients.

The recommendation generation module 218 is configured to generate the one or more medical recommendations corresponding to the predicted CCO based on the received request, the determined set of recovery patterns, the result of classification and the predicted CCO by using the health management based AI model. In an embodiment of the present disclosure, the one or more medical recommendations correspond to supply of oxygen and nutrients to tissue of the one or more patients, extent of cardiac dysfunction, optimal course of therapy, patient progress management, check points for rehabilitation in patient with damaged or diseased heart, fluid status control and the like. In generating one or more medical recommendations corresponding to the predicted CCO based on the received request, the determined set of recovery patterns, the result of classification and the predicted CCO by using the health management based AI model, the recommendation generation module 218 correlates the received request, the determined set of recovery patterns, the result of classification and the predicted CCO by using the health management based AI model. Further, the recommendation generation module 218 generates the one or more medical recommendations based on result of the correlation by using the health management based AI model. In an embodiment of the present disclosure, the one or more medical recommendations are generated to avoid medical risks associated with the one or more patients. For example, the medical risks include stroke, cardiac arrest, peripheral artery disease and the like.

The data output module 220 is configured to output the predicted CCO of the one or more patients and the generated one or more medical recommendations on user interface screen of the one or more electronic devices 102 associated with the one or more patients and the one or more medical professionals.

The model generation module 222 is configured to receive a clinical data associated with a plurality of historical patients with one or more similar patient profiles. In an embodiment of the present disclosure, the clinical data is a time series data collected from the plurality of historical patients during their stay in ICU for training and testing the health management based AI model. The one or more similar patient profiles correspond to patients who exhibit similar behavior or response to medical care provided by the one or more medical professionals. In an embodiment of the present disclosure, a clinical database is created from the received clinical data. In an exemplary embodiment of the present disclosure, the clinical data includes the physiological data, vital signs, demographic details, pretreatment symptoms, treatments, and responses thereto, of the plurality of historical patients. For example, the demographic details include age, race, gender of the patient and the like. In an embodiment of the present disclosure, the plurality of historical patients are patients which are continuously monitored on multiple physiological data and vital signs during their post-surgery recovery in Intensive Care Units (ICUs) to create the clinical database including the clinical data. Further, the model generation module 222 is configured to identify one or more recovery patterns for the one or more similar patient profiles exhibiting similar response to one or more selected treatment regime based on the created clinical database. The model generation module 222 determines behavioral response of CCO of the plurality of historical patients by using the identified one or more recovery patterns. In an embodiment of the present disclosure, the model generation module 222 is configured to create the clinical database including the clinical data captured from the plurality of historical patients having the one or more similar patient profiles and identify the one or more recovery patterns for the one or more similar patient profiles which exhibits similar response to the one or more selected treatment regime, utilizing the one or more recovery patterns for learning the behavioral response of CCO of the plurality of historical patients. Furthermore, the model generation module 222 generates the health management based AI model based on the created clinical database, identified one or more recovery patterns and the determined behavioral response. Further, data that may be used for modeling may not be limited to the clinical data as additional physiological data may also be utilized for further enhancing prediction and accuracy of the health management based AI model. In an embodiment of the present disclosure, the generated health management based AI model enables automated classification of the one or more patients into the one or more predefined profiles from the set of recovery patterns of known symptoms and the one or more responses of the one or more patients to the treatment regime. In an embodiment of the present disclosure, the prediction model is adapted to learn patterns from the physiological data of the one or more patients and identify one or more similar patterns across different patients.

Further, the health management based AI model is adapted to predict or forecast values for continuous stream of data given a past historical trend. The main objective of the health management based AI model is to learn patterns from input training data streams and identify patterns that potentially show similar trends across different patients. In an embodiment of the present disclosure, these trends are not easily identified with simple statistical analysis and there is a need for more complicated models that can learn intricate patterns embedded in time series data. The modeling approach used for generating the health management based AI model is based on regression trees which generates a collection of rules with regression models to generate predictions accurately. In an embodiment of the present disclosure, a tree based rule model learner may also be used to generate rules to predict CCO of the one or more patients.

In an embodiment of the present disclosure, the clinical data is collected for modelling from patients who meet one or more predefined criteria. The one or more predefined criteria include patients with at least 80% of Central Venous Pressure (CVP) or Right Atrial Pressure (RAP) populated for their stay in ICU. Further, the one or more predefined criteria include patients with at least 80% of Aortic Regurgitation (AR) populated for their stay in ICU. The one or more predefined criteria also include patients with at least 80% of Continuous Cardiac Output (CCO) or Cardiac Output (CO) populated for their stay in ICU. In an embodiment of the present disclosure, the clinical data collected from the patients who meet the one or more predefined criteria is utilized for generating the health management based AI model.

Further, the pre-processing module 224 is configured to pre-process the received clinical data of the plurality of historical patients by imputing the received clinical data with linear interpolation for obtaining missing data streams in the received clinical data. In an embodiment of the present disclosure, pre-processing compensates for missing data in the received clinical data due to various operational and sensor issues. The missing data may be either filtered out from analysis or if only a small section of data is missing, then the missing data is imputed using various interpolation techniques. In an embodiment of the present disclosure, the missing data is imputed with linear interpolation. For example, any missing data from a variable, which could account for a maximum of 20% of time series, may be imputed using linear interpolation.

The accuracy determination module 226 is configured to determine accuracy of the predicted CCO of the one or more patients based on regression trees. In an embodiment of the present disclosure, the regression trees generate a collection of rules with regression models to generate predictions accurately. In determining accuracy of the predicted CCO of the one or more patients based on the regression trees, the accuracy determination module 226 splits clinical data into one or more training data sets and one or more testing data sets. Further, the accuracy determination module 226 generates the health management based AI model by using the one or more training data sets. In an embodiment of the present disclosure, the health management based AI model corresponds to rule based model. Furthermore, the accuracy determination module 226 predicts CCO values from the one or more testing data sets by using the generated health management based AI model. The accuracy determination module 226 determines accuracy of the predicted CCO values by comparing the predicted CCO values with the clinical data. For example, 60% of complete data set i.e., the clinical data, is used for learning the health management based AI model and 40% of the clinical data is used for testing the health management based AI model.

The data validation module 228 is configured to validate the accuracy of the predicted CCO values by implementing squared error or correlation metric on the predicted CCO values.

In operation, the plurality of historical patients are continuously monitored on the clinical data during their post-surgery recovery in intensive care units (ICU). Further, inherent patterns are generated based on historical data collected from patients in the past i.e., the clinical data, where such data corresponds to similar patients' profiles that exhibit similar behavior or response to the medical care provided. These patterns are then utilized to generate the health management based AI model of predictive nature which may provide new incoming patients their prognosis into the future. The modeling approach as disclosed herein leads to identification of potentially useful patterns of recovery and further the generated health management based AI model leads to prediction of a patient's condition during recovery. In an embodiment of the present disclosure, the physiological data collected from the one or more patients in the ICU who have undergone cardiac surgery is analyzed. The one or more patients are monitored continuously, and various physiological data is collected on a minute-by-minute basis during their recovery to normality under medical supervision in the ICU. In an embodiment of the present disclosure, the health management based AI model learns the generated inherent patterns to enable automated classification of similar CCO response profiles and enable prediction of CCO ahead of time for new incoming patients whose current physiological data is provided as an input to the health management based AI model.

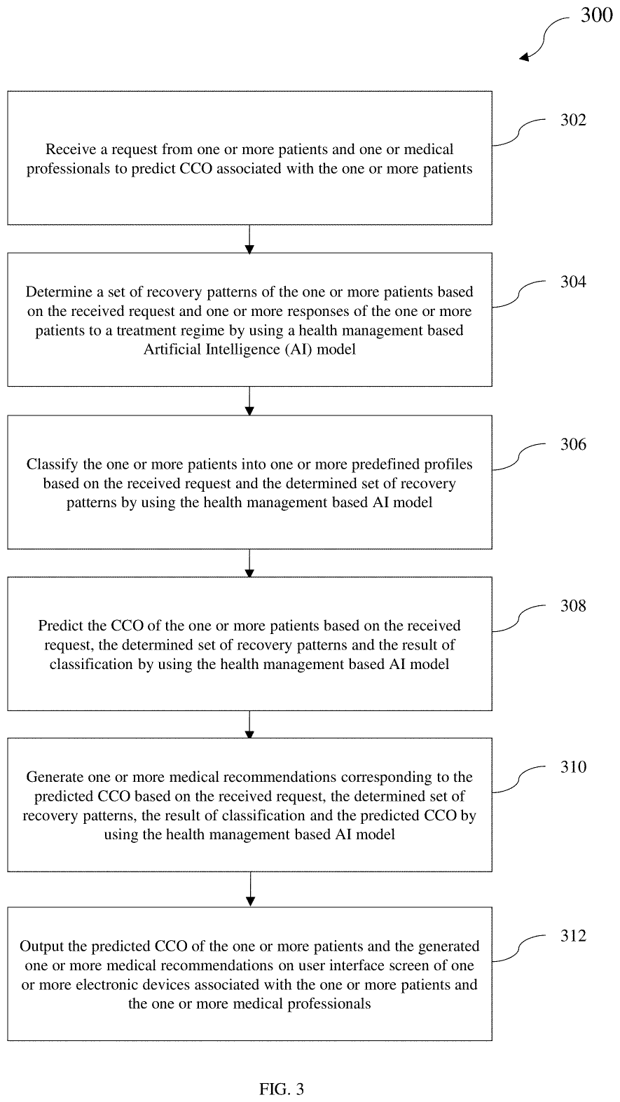

FIG. 3 is a process flow diagram illustrating an exemplary AI based method for predicting CCO of patients, in accordance with an embodiment of the present disclosure. At step 302, a request is received from one or more patients, one or medical professionals or a combination thereof to predict CCO associated with the one or more patients. For example, the one or more medical professionals may be physician, nurse and the like. In an embodiment of the present disclosure, the received request includes physiological data of the one or more patients. The physiological data of the one or more patients include Arterial Pressures (AR), Heart Rate (HR), Central Venous Pressure (CVP), Pulmonary Artery Pressure (PAP), Peripheral capillary oxygen saturation (SpO2), Mixed venous oxygen saturation (SvO2), Core Body Temperature (CBT), Continuous Systemic Vascular Resistance (CSVR) and the like. In an embodiment of the present disclosure, the one or more patients are ICU patients who have undergone cardiac surgery. The one or more patients are monitored continuously, and the physiological data is collected on a minute by minute basis during their recovery to normality under medical supervision in the ICU. Further, the physiological data is captured by a set of physiological sensors 108, such as Electrocardiogram (ECG) sensor, blood pressure sensor, temperature sensor, heart rate sensor, blood glucose sensor and the like. The AR may correspond to Systolic, Diastolic and Mean. In an embodiment of the present disclosure, the request may be received from one or more electronic devices 102 associated with the one or more patients and the one or more medical professionals. In an exemplary embodiment of the present disclosure, the one or more electronic devices 102 may include a laptop computer, desktop computer, tablet computer, smartphone, wearable device, smart watch, and the like.

At step 304, a set of recovery patterns of the one or more patients are determined based on the received request and one or more responses of the one or more patients to a treatment regime by using a health management based Artificial Intelligence (AI) model.

At step 306, the one or more patients are classified into one or more predefined profiles based on the received request and the determined set of recovery patterns by using the health management based AI model. In an embodiment of the present disclosure, each of the one or more predefined profiles corresponds to a set of patients with similar recovery patterns.

At step 308, the CCO of the one or more patients is predicted based on the received request, the determined set of recovery patterns and the result of classification by using the health management based AI model. In an embodiment of the present disclosure, the CCO of the one or more patients is physiological parameter of the one or more patients. In an embodiment of the present disclosure, the CCO of the one or more patients corresponds to CCO level of the one or more patients.

At step 310, one or more medical recommendations corresponding to the predicted CCO are generated based on the received request, the determined set of recovery patterns, the result of classification and the predicted CCO by using the health management based AI model. In an embodiment of the present disclosure, the one or more medical recommendations correspond to supply of oxygen and nutrients to tissue of the one or more patients, extent of cardiac dysfunction, optimal course of therapy, patient progress management, check points for rehabilitation in patient with damaged or diseased heart, fluid status control and the like. In generating one or more medical recommendations corresponding to the predicted CCO based on the received request, the determined set of recovery patterns, the result of classification and the predicted CCO by using the health management based AI model, the AI based method 300 includes correlating the received request, the determined set of recovery patterns, the result of classification and the predicted CCO by using the health management based AI model. Further, the AI based method 300 includes generating the one or more medical recommendations based on result of the correlation by using the health management based AI model. In an embodiment of the present disclosure, the one or more medical recommendations are generated to avoid medical risks associated with the one or more patients. For example, the medical risks include stroke, cardiac arrest, peripheral artery disease and the like.

At step 312, the predicted CCO of the one or more patients and the generated one or more medical recommendations are outputted on user interface screen of the one or more electronic devices 102 associated with the one or more patients and the one or more medical professionals.

Further, the AI based method 300 includes receiving a clinical data associated with a plurality of historical patients with one or more similar patient profiles. In an embodiment of the present disclosure, the clinical data is a time series data collected from the plurality of historical patients during their stay in ICU for training and testing the health management based AI model. The one or more similar patient profiles correspond to patients who exhibit similar behavior or response to medical care provided by the one or more medical professionals. In an embodiment of the present disclosure, a clinical database is created from the received clinical data. In an exemplary embodiment of the present disclosure, the clinical data includes physiological data, vital signs, demographic details, pretreatment symptoms, treatments, and responses thereto, of the plurality of historical patients. For example, the demographic details include age, race, gender of the patient and the like. In an embodiment of the present disclosure, the plurality of historical patients are patients which are continuously monitored on multiple physiological data and vital signs during their post-surgery recovery in Intensive Care Units (ICUs) to create the clinical database including the clinical data. Further, the AI based method 300 includes identifying one or more recovery patterns for the one or more similar patient profiles exhibiting similar response to one or more selected treatment regime based on the created clinical database. The AI based method 300 includes determining behavioral response of CCO of the plurality of historical patients by using the identified one or more recovery patterns. In an embodiment of the present disclosure, the AI based method 300 includes creating the clinical database including the clinical data captured from the plurality of historical patients having the one or more similar patient profiles and identifying the one or more recovery patterns for the one or more similar patient profiles which exhibits similar response to the one or more selected treatment regime, utilizing the one or more recovery patterns for learning the behavioral response of CCO of the plurality of historical patients. Furthermore, the AI based method 300 includes generating the health management based AI model based on the created clinical database, identified one or more recovery patterns and the determined behavioral response. Further, data that may be used for modeling may not be limited to the clinical data as additional physiological data may also be utilized for further enhancing prediction and accuracy of the health management based AI model. In an embodiment of the present disclosure, the generated health management based AI model enables automated classification of the one or more patients into the one or more predefined profiles from the set of recovery patterns of known symptoms and the one or more responses of the one or more patients to the treatment regime. In an embodiment of the present disclosure, the prediction model is adapted to learn patterns from the physiological data of the one or more patients and identify one or more similar patterns across different patients.

Furthermore, the health management based AI model is adapted to predict or forecast values for continuous stream of data given a past historical trend. The main objective of the health management based AI model is to learn patterns from input training data streams and identify patterns that potentially show similar trends across different patients. In an embodiment of the present disclosure, these trends are not easily identified with simple statistical analysis and there is a need for more complicated models that can learn intricate patterns embedded in time series data. The modeling approach used for generating the health management based AI model is based on regression trees which generates a collection of rules with regression models to generate predictions accurately. In an embodiment of the present disclosure, a tree based rule model learner may also be used to generate rules to predict CCO of the one or more patients.

In an embodiment of the present disclosure, the clinical data is collected for modelling from patients who meet one or more predefined criteria. The one or more predefined criteria include patients with at least 80% of Central Venous Pressure (CVP) or Right Atrial Pressure (RAP) populated for their stay in ICU. Further, the one or more predefined criteria include patients with at least 80% of Aortic Regurgitation (AR) populated for their stay in ICU. The one or more predefined criteria also include patients with at least 80% of Continuous Cardiac Output (CCO) or Cardiac Output (CO) populated for their stay in ICU. In an embodiment of the present disclosure, the clinical data collected from the patients who meet the one or more predefined criteria is utilized for generating the health management based AI model.

Further, the AI based method 300 includes pre-processing the received clinical data of the plurality of historical patients by imputing the received clinical data with linear interpolation for obtaining missing data streams in the received clinical data. In an embodiment of the present disclosure, pre-processing compensates for missing data in the received clinical data due to various operational and sensor issues. The missing data may be either filtered out from analysis or if only a small section of data is missing, then the missing data is imputed using various interpolation techniques. In an embodiment of the present disclosure, the missing data is imputed with linear interpolation. For example, any missing data from a variable, which could account for a maximum of 20% of time series, may be imputed using linear interpolation.

Furthermore, the AI based method 300 includes determining accuracy of the predicted CCO of the one or more patients based on regression trees. In an embodiment of the present disclosure, the regression trees generate a collection of rules with regression models to generate predictions accurately. In determining accuracy of the predicted CCO of the one or more patients based on the regression trees, the AI based method 300 includes splitting clinical data into one or more training data sets and one or more testing data sets. Further, the AI based method 300 includes generating the health management based AI model by using the one or more training data sets. In an embodiment of the present disclosure, the health management based AI model corresponds to rule based model. Furthermore, the AI based method 300 includes predicting CCO values from the one or more testing data sets by using the generated health management based AI model. The AI based method 300 includes determining accuracy of the predicted CCO values by comparing the predicted CCO values with the clinical data. For example, 60% of complete data set i.e., the clinical data, is used for learning the health management based AI model and 40% of the clinical data is used for testing the health management based AI model.

Further, the AI based method 300 includes validating the accuracy of the predicted CCO values by implementing squared error or correlation metric on the predicted CCO values.

The AI based method 300 may be implemented in any suitable hardware, software, firmware, or combination thereof.

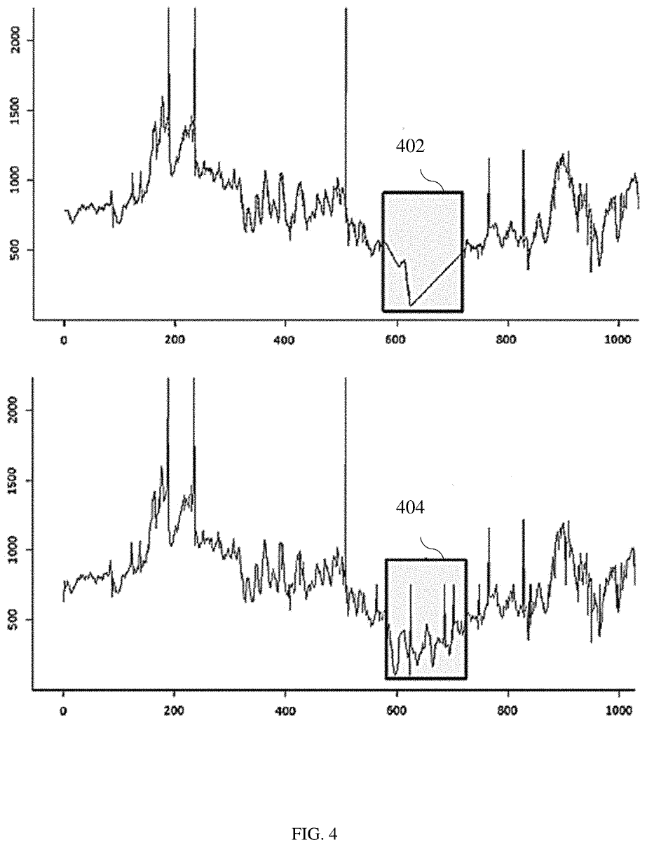

FIG. 4 is an exemplary graphical representation illustrating a sample time series for comparing nearest neighbour interpolation and linear interpolation to represent missing data replacement, in accordance with an embodiment of the present disclosure. As shown in FIG. 4, data imputation using a nearest calculated clinical data value is performed to fill in the missing data streams for short sections of missing data. The interpolation for nearest neighbour is done by comparing all the physiological variables data among all the patients that was collected for creating the health management based AI model. Further, FIG. 4 also shows linear interpolation approximation 402 and nearest neighbour approximation 404.

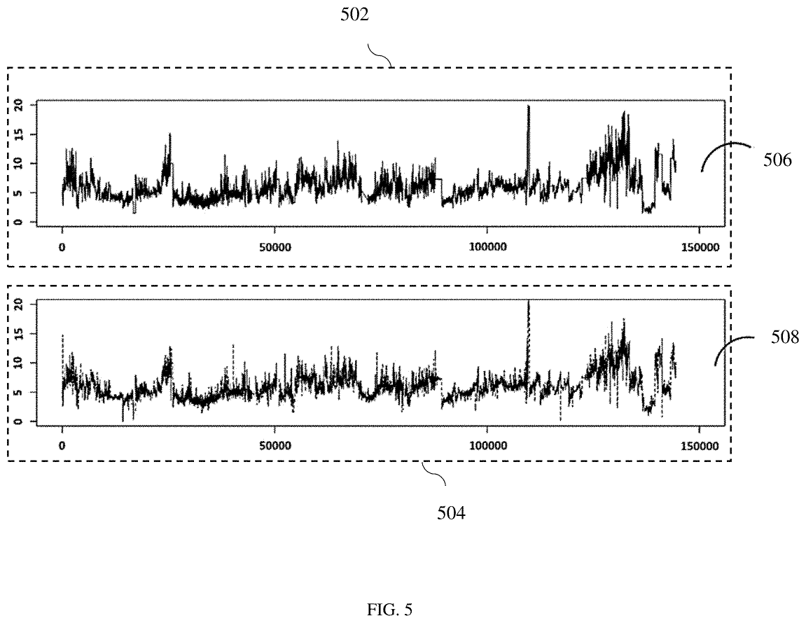

FIG. 5 is an exemplary plot diagram illustrating a health management based AI model prediction of CCO 10 minutes into future based on input training data, in accordance with an embodiment of the present disclosure. As shown in FIG. 5, 502 depicts actual CCO output and 504 depicts health management based AI model's CCO output. Further, the health management based AI model is trained on input data to learn forecasted output of CCO 10 Minutes into the future. In an embodiment of the present disclosure, first plot 506 represents actual value of CCO to be predicted and second plot 508 represents output of trained health management based AI model prediction of CCO. The plot clearly illustrates that the health management based AI model is able to accurately learn the recovery patterns for predicting CCO from the training data.

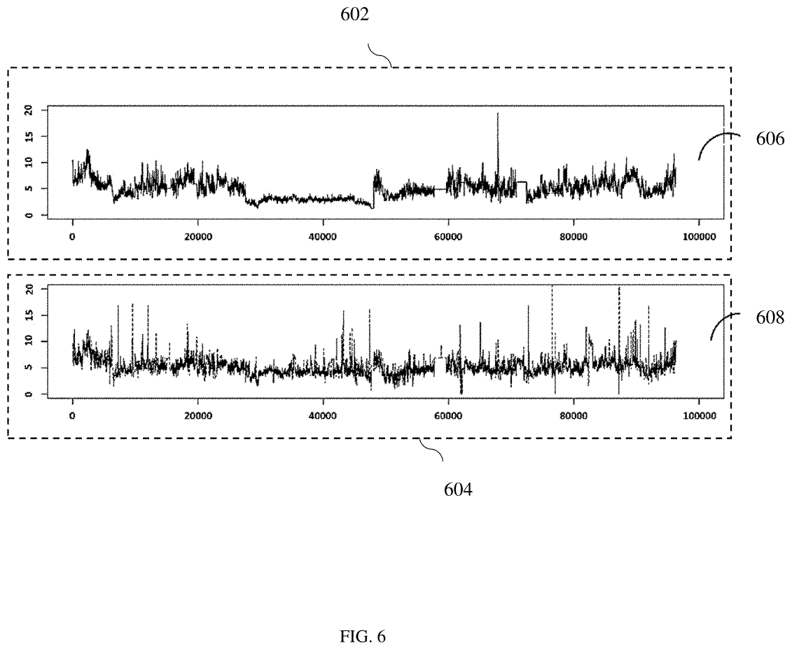

FIG. 6 is an exemplary plot diagram illustrating a prediction of CCO 10 minutes into future with testing data to validate the health management based AI model, in accordance with an embodiment of the present disclosure. As shown in FIG. 6, 602 depicts actual CCO output and 604 depicts health management based AI model's CCO output. Further, first plot 606 represents value of CCO to be predicted for 10 Minutes into future and second plot 608 represents actual value of CCO predicted by the health management based AI model. The health management based AI model utilizes the recovery patterns learned from the training data and provide accurate predictions of CCO, as shown in FIG. 6.

Further, the plots 606, 608 of FIG. 6 clearly depicts that most of times the predictions are close to the actual values of CCO. In some cases, the actual predicted values are offset with a certain deviation, nonetheless it follows the trend of upward and downward movement of actual CCO values. This is vital for the physician to understand the condition of the patient, which is accurately provided by the health management based AI model herein.

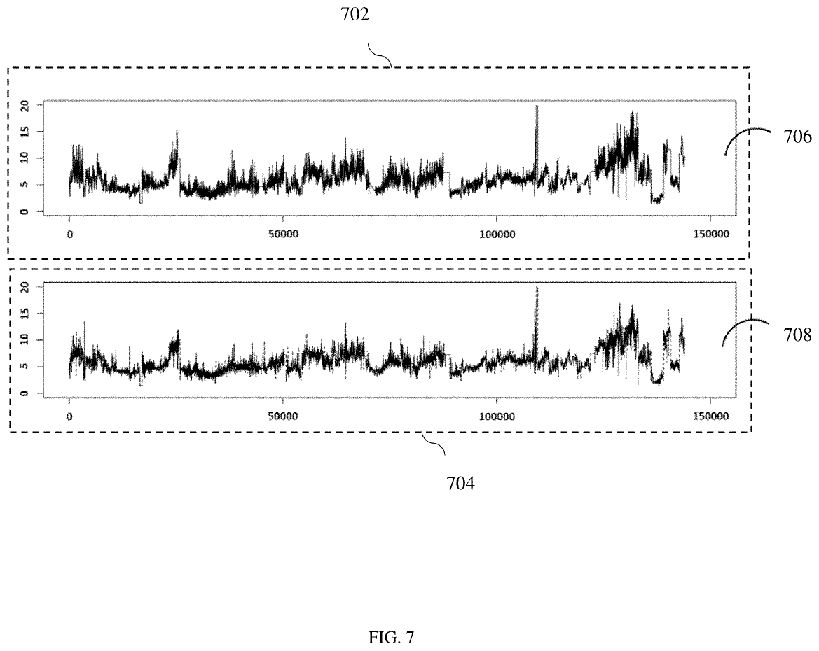

FIG. 7 is an exemplary plot diagram illustrating the health management based AI model prediction of CCO 30 minutes into future based on the input training data, in accordance with another embodiment of the present disclosure. As shown in FIG. 7, 702 depicts actual CCO output and 704 depicts health management based AI model's CCO output. Further, the health management based AI model is modified to predict ahead of time for 30 minutes into future the values of CCO from the current physiological readings. The plot shows the test predictions of CCO 30 minutes into future compared with original data. Furthermore, first plot 706 represents actual value of CCO (training data) to be predicted and second plot 708 represents trained health management based AI model predictions on the training data.

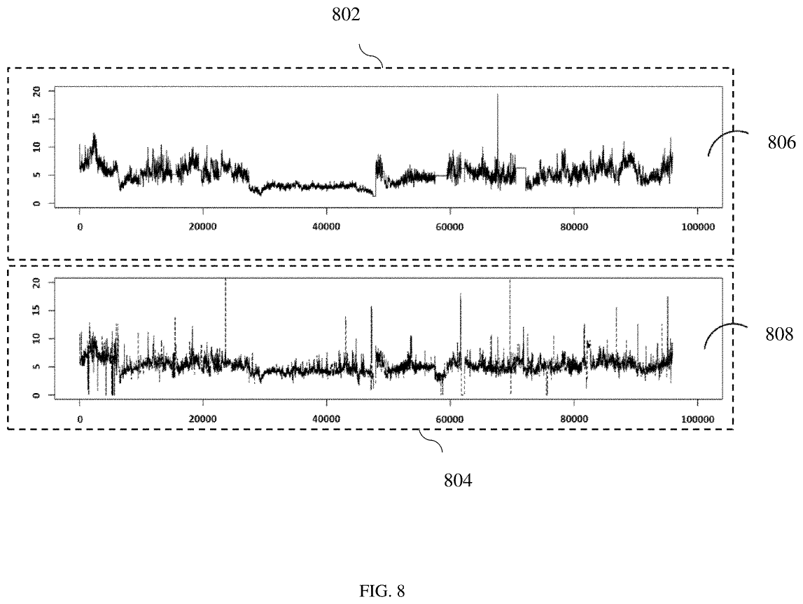

FIG. 8 is an exemplary plot diagram illustrating prediction of CCO 30 minutes into future by the health management based AI model with the testing data to validate the health management based AI model, in accordance with another embodiment of the present disclosure. As shown in FIG. 8, 802 depicts actual CCO output and 804 depicts health management based AI model's CCO output. Further, first plot 806 represents actual value of CCO 30 minutes into the future, which is to be predicted and second plot 808 represents the value of CCO predicted by the health management based AI model. From plots 806, 808 it can be clearly seen that predicting CCO further into the future is difficult and hence there is a slight deterioration in the output accuracy of actual CCO values but the trend of CCO movement is still predicted with a high degree of accuracy.

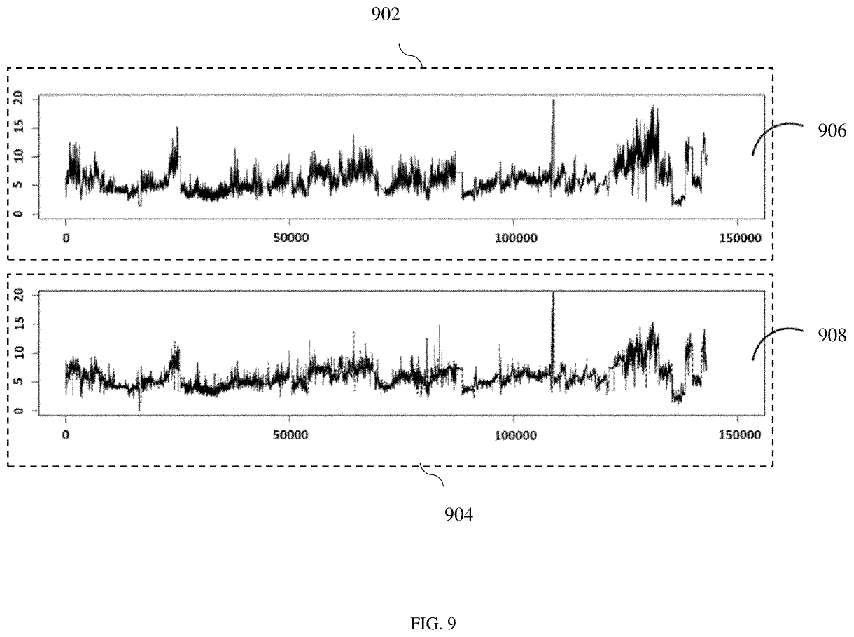

FIG. 9 is an exemplary plot diagram illustrating the health management based AI model prediction of CCO 60 minutes into future based on the input training data, in accordance with an embodiment of the present disclosure. As shown in FIG. 9, 902 depicts actual CCO output and 904 depicts health management based AI model's CCO output. Further, the plot diagram illustrates that the health management based AI model is being trained to learn the forecasted output of CCO 60 Minutes into future and shows trained health management based AI model prediction based on the input training data. Furthermore, first plot 906 represents actual value of CCO training data to be predicted and second plot 908 represents the trained health management based AI model predictions of CCO.

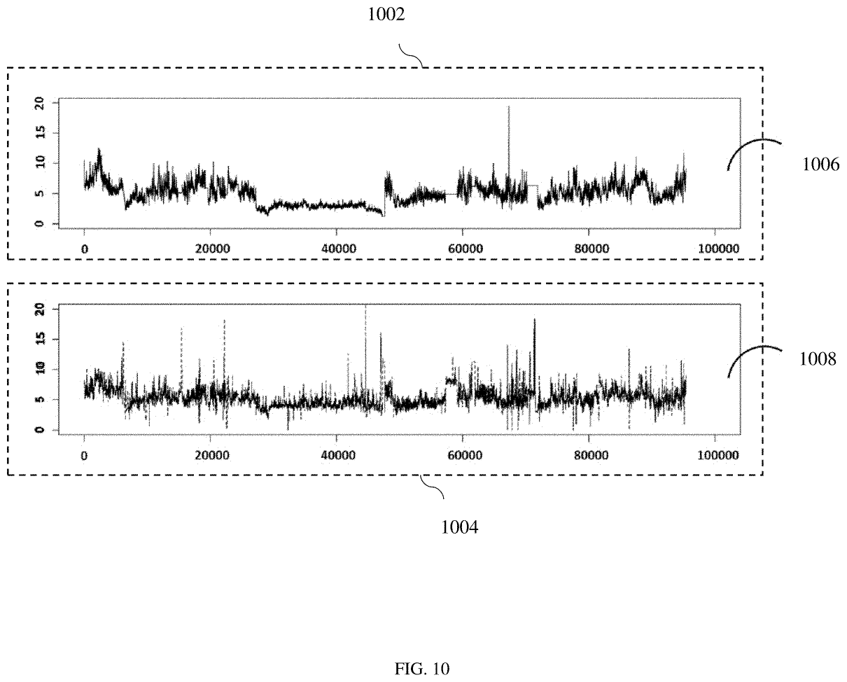

FIG. 10 is an exemplary plot diagram illustrating prediction of CCO 60 Minutes into future by the health management based AI model with the testing data to validate the health management based AI model, in accordance with an embodiment of the present disclosure. As shown in FIG. 10, 1002 depicts actual CCO output and 1004 depicts health management based AI model's CCO output. Further, first plot 1006 represents value of CCO 60 Minutes into future, which is to be predicted and the second plot 1008 represents actual value of CCO predicted by the health management based AI model. From plots 1006, 1008 it may be clearly seen that there is further deterioration in the output accuracy of actual CCO values but the trend of CCO movement is still predicted with a high degree of accuracy. In exemplary embodiments as disclosed herein indicates that the CCO in the near future for 10, 30 and 60 minutes is accurately estimated and trending direction of CCO is precisely identified which may aid in better prognosis of patients ahead of time for preventive care.

Thus, various embodiments of the present AI based computing system 104 provide a solution to predict Continuous Cardiac Output (CCO) of patients. The AI based computing system 104 creates the health management based AI model to predict the Continuous Cardiac Output (CCO) of the one or more patients in near future based on the physiological data. Further, the health management based AI model accurately assess condition of the one or more patients ahead of time. In an embodiment of the present disclosure, the AI based computing system 104 predicts the physiological condition of the one or more patients ahead of time using the clinical data during post-surgery recovery in Intensive Care Unit (ICU). The AI based computing system 104 pre-process the clinical data by imputing with linear interpolation to obtain missing data streams in the clinical data and determine the accuracy of the predicted CCO values by comparing an output of the health management based AI model with the clinical data. Further, the AI based computing system 104 predict future values of continuous cardiac output of the one or more patients under observation in ICU from a plurality of physiological parameters using the health management based AI model. In an embodiment of the present disclosure, the AI based computing system 104 accurately estimates the CCO in the near future for 10, 30 and 60 minutes and precisely identify trending direction of CCO which may aid in better prognosis of patients ahead of time for preventive care.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The CPUs are interconnected via system bus 208 to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limiting, of the scope of the invention, which is set forth in the following claims.

We claim:

1. An Artificial Intelligence (AI) based computing system for predicting Continuous Cardiac Output (CCO) of patients, the computing system comprising:
one or more hardware processors; and
a memory coupled to the one or more hardware processors, wherein the memory comprises a plurality of modules in the form of programmable instructions executable by the one or more hardware processors, wherein the plurality of modules comprises:
a data receiver module configured to receive a request from at least one of: one or more patients and one or medical professionals to predict CCO associated with the one or more patients, wherein the received request comprises: physiological data of the one or more patients;
a pattern determination module configured to determine a set of recovery patterns of the one or more patients based on the received request and one or more responses of the one or more patients to a treatment regime by using a health management based AI model;
a patient classification module configured to classify the one or more patients into one or more predefined profiles based on the received request and the determined set of recovery patterns by using the health management based AI model, wherein each of the one or more predefined profiles corresponds to a set of patients with similar recovery patterns;
a data prediction module configured to predict the CCO of the one or more patients based on the received request, the determined set of recovery patterns and the result of classification by using the health management based AI model;
a recommendation generation module configured to generate one or more medical recommendations corresponding to the predicted CCO based on the received request, the determined set of recovery patterns, the result of classification and the predicted CCO by using the health management based AI model, wherein the one or more medical recommendations correspond to supply of oxygen and nutrients to tissue of the one or more patients, extent of cardiac dysfunction, optimal course of therapy, patient progress management, check points for rehabilitation in patient with one of: damaged and diseased heart and fluid status control; and
a data output module configured to output the predicted CCO of the one or more patients and the generated one or more medical recommendations on user interface screen of one or more electronic devices associated with the one or more patients and the one or more medical professionals.

2. The AI based computing system of claim 1, wherein the physiological data of the one or more patients comprises: Arterial Pressures (AR), Heart Rate (HR), Central Venous Pressure (CVP), Pulmonary Artery Pressure (PAP), Peripheral capillary oxygen saturation (SpO2), Mixed venous oxygen saturation (SvO2), Core Body Temperature (CBT) and Continuous Systemic Vascular Resistance (CSVR).

3. The AI based computing system of claim 1, further comprises a model generation module configured to:
receive a clinical data associated with a plurality of historical patients with one or more similar patient profiles, wherein a clinical database is created from the received clinical data;
identify one or more recovery patterns for the one or more similar patient profiles exhibiting similar response to one or more selected treatment regime based on the created clinical database;
determine behavioral response of CCO of the plurality of historical patients by using the identified one or more recovery patterns; and
generate the health management based AI model based on the created clinical database, identified one or more recovery patterns and the determined behavioral response, wherein the generated health management based AI model enables automated classification of the one or more patients into the one or more predefined profiles from the set of recovery patterns of known symptoms and the one or more responses of the one or more patients to the treatment regime.

4. The AI based computing system of claim 3, wherein the clinical data comprise: physiological data, vital signs, demographic details, pretreatment symptoms, treatments, and responses thereto, of the plurality of historical patients.

5. The AI based computing system of claim 4, wherein the demographic details comprise: age, race and gender of the patient.

6. The AI based computing system of claim 3, further comprises a pre-processing module configured to pre-process the received clinical data of the plurality of historical patients by imputing the received clinical data with linear interpolation for obtaining missing data streams in the received clinical data.

7. The AI based computing system of claim 1, further comprises an accuracy determination module configured to determine accuracy of the predicted CCO of the one or more patients based on regression trees, wherein the regression trees generate a collection of rules with regression models to generate predictions accurately.

8. The AI based computing system of claim 7, wherein in determining accuracy of the predicted CCO of the one or more patients based on the regression trees, the accuracy determination module is configured to:
split clinical data into one or more training data sets and one or more testing data sets;
generate the health management based AI model by using the one or more training data sets, wherein the health management based AI model corresponds to rule based model;
predict CCO values from the one or more testing data sets by using the generated health management based AI model; and
determine accuracy of the predicted CCO values by comparing the predicted CCO values with the clinical data.

9. The AI based computing system of claim 8, further comprises a data validation module configured to validate the accuracy of the predicted CCO values by implementing one of a: squared error and correlation metric on the predicted CCO values.

10. The AI based computing system of claim 1, wherein in generating one or more medical recommendations corresponding to the predicted CCO based on the received request, the determined set of recovery patterns, the result of classification and the predicted CCO by using the health management based AI model, the recommendation generation module is configured to:
correlate the received request, the determined set of recovery patterns, the result of classification and the predicted CCO by using the health management based AI model; and
generate the one or more medical recommendations based on result of the correlation by using the health management based AI model.

11. An Artificial Intelligence (AI) based method for predicting Continuous Cardiac Output (CCO) of patients, the AI based method comprising:
receiving, by one or more hardware processors, a request from at least one of: one or more patients and one or more medical professionals to predict CCO associated with the one or more patients, wherein the received request comprises: physiological data of the one or more patients;
determining, by the one or more hardware processors, a set of recovery patterns of the one or more patients based on the received request and one or more responses of the one or more patients to a treatment regime by using a health management based AI model;
classifying, by the one or more hardware processors, the one or more patients into one or more predefined profiles based on the received request and the determined set of recovery patterns by using the health management based AI model, wherein each of the one or more predefined profiles corresponds to a set of patients with similar recovery patterns;
predicting, by the one or more hardware processors, the CCO of the one or more patients based on the received request, the determined set of recovery patterns and the result of classification by using the health management based AI model;
generating, by the one hardware processors, one or more medical recommendations corresponding to the predicted CCO based on the received request, the determined set of recovery patterns, the result of classification and the predicted CCO by using the health management based AI model, wherein the one or more medical recommendations correspond to supply of oxygen and nutrients to tissue of the one or more patients, extent of cardiac dysfunction, optimal course of therapy, patient progress management, check points for rehabilitation in patient with one of: damaged and diseased heart and fluid status control; and
outputting, by the one or more hardware processors, the predicted CCO of the one or more patients and the generated one or more medical recommendations on user interface screen of one or more electronic devices associated with the one or more patients and the one or more medical professionals.

12. The AI based method of claim 11, wherein the physiological data of the one or more patients comprises: Arterial Pressures (AR), Heart Rate (HR), Central Venous Pressure (CVP), Pulmonary Artery Pressure (PAP), Peripheral capillary oxygen saturation (SpO2), Mixed venous oxygen saturation (SvO2), Core Body Temperature (CBT) and Continuous Systemic Vascular Resistance (CSVR).

13. The AI based method of claim 11, further comprises:
receiving a clinical data associated with a plurality of historical patients with one or more similar patient profiles, wherein a clinical database is created from the received clinical data;
identifying one or more recovery patterns for the one or more similar patient profiles exhibiting similar response to one or more selected treatment regime based on the created clinical database;
determining behavioral response of CCO of the plurality of historical patients by using the identified one or more recovery patterns; and
generating the health management based AI model based on the created clinical database, identified one or more recovery patterns and the determined behavioral response, wherein the generated health management based AI model enables automated classification of the one or more patients into the one or more predefined profiles from the set of recovery patterns of known symptoms and the one or more responses of the one or more patients to the treatment regime.

14. The AI based method of claim 13, wherein the clinical data comprise: physiological data, vital signs, demographic details, pretreatment symptoms, treatments, and responses thereto, of the plurality of historical patients.

15. The AI based method of claim 14, wherein the demographic details comprise: age, race and gender of the patient.

16. The AI based method of claim 13, further comprises pre-processing the received clinical data of the plurality of historical patients by imputing the received clinical data with linear interpolation for obtaining missing data streams in the received clinical data.

17. The AI based method of claim 11, further comprises determining accuracy of the predicted CCO of the one or more patients based on regression trees, wherein the regression trees generate a collection of rules with regression models to generate predictions accurately.

18. The AI based method of claim 17, wherein determining accuracy of the predicted CCO of the one or more patients based on the regression trees comprise:
splitting clinical data into one or more training data sets and one or more testing data sets;
generating the health management based AI model by using the one or more training data sets, wherein the health management based AI model corresponds to rule based model;
predicting CCO values from the one or more testing data sets by using the generated health management based AI model; and
determining accuracy of the predicted CCO values by comparing the predicted CCO values with the clinical data.

19. The AI based method of claim 18, further comprises validating the accuracy of the predicted CCO values by implementing one of a: squared error and correlation metric on the predicted CCO values.

20. The AI based method of claim 11, wherein generating one or more medical recommendations corresponding to the predicted CCO based on the received request, the determined set of recovery patterns, the result of classification and the predicted CCO by using the health management based AI model comprises:
correlating the received request, the determined set of recovery patterns, the result of classification and the predicted CCO by using the health management based AI model; and
generating the one or more medical recommendations based on result of the correlation by using the health management based AI model.
